(19) United States
(12) Patent Application Publication
(10) Pub. No.: US 2009/0029856 A1
(43) Pub. Date: Jan. 29, 2009

(54) Hinokitiol As A Plant Pesticide

(75) Inventors: Cruz Avila-Adame, Davis, CA (US); Huazhang Huang, Davis, CA (US); Janet Hernandez, West Sacramento, CA (US); Brian Campbell, Davis, CA (US); Lorena Fernandez, Sacramento, CA (US); Pamela G. Marrone, Davis, CA (US); Marja Koivunen, Davis, CA (US)

Correspondence Address: CHERYL H AGRIS PHD, PO BOX 806, PELHAM, NY, 10803, US

(73) Assignee: Marrone Organic Innovations, Inc., Davis, CA (US)

(21) Appl. No.: 12/178,594

(22) Filed: Jul. 23, 2008

Related U.S. Application Data
(60) Provisional application No. 60/951,520, filed on Jul. 24, 2007.
(60) Provisional application No. 60/951,523, filed on Jul. 24, 2007.

(51) Int. Cl. A01N 35/00; A61K 31/122; A01P 1/00; A01P 3/00; A01P 13/00

(52) U.S. Cl. 504/118; 514/675; 504/348

(57) ABSTRACT

The present invention discloses that a natural tropolone compound, hinokitiol, also known as β-Thujaplicin as an effective biopesticide for use in controlling plant pests. In particular, hinokitiol and/or salts thereof may be used in modulating non-Erwinia plant microbial infection or in the formulation of a non-Erwinia antimicrobial agent. Alternatively, hinokitiol may be used as a natural herbicide to control the germination and growth of weeds. The formulation used in the present invention may contain a chelator, pH adjustor and UV protection agent in an amount effective to stabilize said hinokitiol.

### PRIORITY CLAIM

This application claims priority under 35 USC 119(e) to US provisional application Ser. No. 60/951,520, filed Jul. 24, 2007 and application Ser. No. 60/951,523, filed Jul. 24, 2007, the contents of which are incorporated herein by reference.

### FIELD OF THE INVENTION

This invention relates to compositions and methods for controlling plant pests, particularly plant pathogenic bacteria, fungi and/or weeds using compounds comprising hinokitiol (β-Thujaplicin) as an active ingredient.

### BACKGROUND OF THE INVENTION

Outbreaks of diseases and pests are commonly observed in plants. Fungal infection and herbicide infestation are two types of plant pests of particular concern.

### Plant Pest Control

Biological control of plant pathogens using microbes, microbial metabolites and other natural products has become important in recent years. There are already a number of microbial products on the market for controlling plant pathogenic fungi (Mycostop®, Serenade®, Sonata®, Aspire®, Primastop®), but there is still a need for natural antifungal compounds for organic farming—products that are safe for both the user and the environment.

Organic growers have discovered that corn gluten meal (a by-product in the manufacture of cornstarch) can serve as an effective pre-emergence herbicide. Since corn gluten meal affects only sprouting seeds, it is safe to use around mature or established plants. Herbicidal “soaps” and plant extracts serve as organic post-emergence herbicides. These products contain compounds with low toxicity, and they are generally degraded fast in the environment. Commercially available post-emergence herbicides include lemongrass oil and d-limonene.

### Hinokitiol

Hinokitiol (4-isopropyl tropolone, MW 164.2), also known, as β-Thujaplicin is an important tropolone compound. It is well known that hinokitiol exhibits strong antimicrobial actions. However, hinokitiol is completely unstable under almost all environmental conditions such as light, heat, and/or their combinations. Hinokitiol also has limited solubility in water.

### SUMMARY OF THE INVENTION

The present invention discloses the use of hinokitiol as a biocontrol agent against plant pests, particularly plant pathogenic bacteria, fungi and/or as a pre- and post-emergence herbicide against weeds. One object of the invention is to provide novel antimicrobial, particularly, antifungal compositions against plant pathogenic fungi that contain hinokitiol as an active ingredient. Another object is to provide a safe, food-grade, non-toxic antimicrobial composition and a method that will not harm the environment. A further object of the invention is to provide novel compositions against both broadleaf and grass weeds that contain hinokitiol as an active ingredient. Another object is to provide a safe, food-grade, non-toxic herbicidal composition and a method that will not harm the environment. The above and other objects are accomplished by the present invention.

Additionally, the invention is directed to compositions containing (a) hinokitiol and/or salts thereof; (b) certain carriers and/or diluents and (c) optionally one or more chelators, UV protection agents and/or basic pH stabilizers. This composition, in a particular embodiment may be used to control plant pathogenic microbes, particularly, plant pathogenic fungi or plant pathogenic bacteria or more particularly, plant pathogenic non-Erwinia bacteria in plants, soil and other growth media and/or to control the germination and growth of weeds. In a particular embodiment, the hinokitiol in the composition is dissolved in a diluent comprising an organic solvent such as an aliphatic carboxylic acid, including but not limited to C1-10 carboxylic acid, e.g., formic acid, acetic acid, propionic acid; an aliphatic alcohol, e.g., methanol, ethanol, isopropanol, butanol; or an aliphatic ketone such as acetone, methyl ethyl ketone, methyl isobutyl ketone. The composition may further comprise a diol such as 1,4-butanediol, 1,6-hexanediol, ethylene glycol, propylene glycol.

The hinokitiol salts in the composition and method of the present invention include but are not limited to sodium, magnesium, copper, calcium, barium, zinc, calcium, tin, cobalt, titanium and vanadium for both herbicide and antimicrobial formulations.

The compositions of the present invention may be in the form of an emulsifiable concentrate (EC), microemulsion (ME), soluble liquid (SL), Emulsion oil-in-water (EW) suspension concentration (SC), wettable granule (WG), or wettable powder (WP), Microencapsulated Suspension.

The invention is also directed to a method of modulating non-Erwinia, plant microbial infection in a plant comprising applying to said plant and/or seeds thereof and/or substrate used for growing said plant an amount of hinokitiol and/or salt thereof or composition of the present invention effective to modulate non-Erwinia plant microbial infection infection in said plant. The term “modulate” is used to mean alter the amount of non-Erwinia plant microbial infection or rate of spread of non-Erwinia plant microbial infection. In a particular embodiment, the hinokitiol is applied in an amount of about 0.005 mg/ml to about 1.5 mg/ml. In a more particular embodiment, the hinokitiol is applied in an amount of about 0.01 to about 1.0 mg/ml. In yet a more particular embodiment, the hinokitiol is applied in an amount of about 0.1 to about 1.0 mg/ml. In a particular embodiment, the plant pathogen is a fungus; in another particular embodiment, the plant pathogen is a non-Erwinia bacteria. In most particular embodiments, the fungus is a Fusarium sp., Botrytis sp., Monilinia sp., Colletotrichum sp, Verticillium sp.; Microphomina sp., and Phytophtora sp, Mucor, Rhizoctonia, Geotrichum, Phoma, and Penicillium. In another most particular embodiment, the bacteria is Xanthomonas.

The invention is further directed to the use of hinokitiol in the preparation of a formulation or composition for use as a non-Erwinia plant antimicrobial agent, particularly, a biofungicide or non-Erwinia antibacterial agent, more particularly a pre-harvest biofungicide either alone or in combination with a non-hinokitiol fungicide or hon-hinokitiol antibacterial agent. They include, but not limited to, the following fungicides such as azoxystrobin, azoxystrobin combination, boscalid, bacillus subtilis, copper sulfate, chlorothalonil, copper hydroxide, cymoxanil, dimethomorph, dechloropropene, fosetyl-aluminum, fludioxonil, fenamidone, iprodione, mefenoxam, mancozeb, metalaxyl, metam sodium, potassium bicarbonate, pyraclostrobin, propiconazole, propicocarb, thiram, thiabendazole, thiophanate-methyl, trifloxystrobin, vinclozolin, sulfur, ziram or the following antibacterial agent such as streptomycin or oxytetracycline.

In yet another embodiment, the invention is directed to a method for modulating growth of monocotyledonous or dicotyledonous weeds comprising applying to said weeds an amount of hinokitiol and/or salt thereof or composition of the present invention effective to modulate growth of said weeds. In a particular embodiment, hinokitiol is applied in an amount ranging from 1 mg/ml to about 50 mg/ml or to about 75 mg/ml. In a more particular embodiment, hinokitiol is applied in an amount ranging from about 2.0 mg/ml to about 10 mg/ml. In a particular embodiment, the hinokitiol is applied to the leaves, stems, flowers, foliage and/or roots of said weeds. In yet a more particular embodiment, the hinokitiol or salt thereof used in the method of the present invention is formulated into the composition of the present invention.

The invention is further directed to the use of hinokitiol in the preparation of a formulation for use as a pre-emergence or post-emergence herbicide, in particular, to control both broad-leaved and grass weeds. The invention is also directed to the use of hinokitiol as a pre or post-emergence herbicide applied in a solvent-based solution such as acetone, ethanol, formic acid, or propionic acid either alone or in combination with another bioherbicide and/or a chemical herbicide. They include, but are not limited to the following herbicides such as lemongrass oil, d-limonene, dichlorophenoxycetic acid (2,4-D), 2,4-D combinations, acrolein, amitrole, bromacil, bromoxynil, chlorsulfuron, clethodim, clopyralid, complexed copper, dicamba, dichlobenil, diquat, diuron, DSMA, endothall, fluazifop-P-butyl, fluridone, fosamine, glufosinate, glyphosate, growth retardant dye, hexazinone, imazapyr, isoxaben, metsulfuron methyl, norflurazon, paraquat, pendimethalin, picloram, prometon, simazine, sulfometuron methyl, tebuthiuron, triclopyr.

### DETAILED DESCRIPTION OF THE INVENTION

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Hinokitiol utilized in this invention may be derived from conventional sources, for example from essential oil of Taiwan hinoki cypress (Chamaecyparis taiwanensis) and Aomori hiba (Thujopsis dolabrata) (natural products), by chemical synthesis (synthesized product) (see, for example, U.S. Pat. Nos. 6,183,748, 6,310,255 and 6,391,347) or can be purchased from commercial sources.

In one embodiment, the present invention provides a composition, particularly a biantimicrobial, more particularly, a bioantibacterial, biofungicidal or bioherbicidal composition comprising, in admixture with a suitable carrier and optionally with a suitable surface active ingredient, hinokitiol. In a particular embodiment, the active ingredient, hinokitiol, is present in the composition of the present invention in the amount of about 0.001% by weight to about 70% by weight and more preferably, between 1-50% by weight. The composition may comprise hinokitiol and/or hinokitiol salts.

The compositions of the present invention may be sprayed on the plant. Alternatively, a plant may be soaked in a solution or immersed in a formulation comprising hinokitiol and/or salt thereof. The compositions of the present invention may be applied to the substrate used to grow the plant, which may include but is not limited to soil, peat moss, sand, agar suspension. Particular embodiments are described in the Examples, infra. These compositions may be in the form of dust, coarse dust, micro granules, granules, wettable powder, emulsifiable concentrate, liquid preparation, suspension concentrate, water degradable granules or oil suspension. In a specific embodiment, the compositions are in solid form in the form of a granule.

The compositions of the present invention may comprise one or more chelators. These include but are not limited to acid chelators from EPA Lists 4A and 4B (Http://www.epa.gov/opprd001/inerts/oldlists.html). These include but are not limited to citric acid, citric acid disodium salt, EDTA disodium salt, metal ions such as zinc, copper, iron, calcium, magnesium, barium, tin, cobalt, titanium and vanadium, phosphonate and aminophosphonate, ascorbic acid and a combination of any of these. The ratio of chelators over hinokitiol ranges from 5:1 to 1:5. The preferred ration depends on chelators, but ratio follows between 2:1 and 1:2. The chelator(s) are present in an amount effective to stabilize said hinokitiol and/or salt thereof. As defined herein, “stabilize” means to prevent degradation of hinokitiol under sunlight and/or temperature at 4° C. and 54° C. for at least 14 days.

The compositions of the present invention may further comprise one or more UV protection agents from EPA List A or B. The content of UV protection agents contains 0.1% to 30%, but preferred content is 0.1% to 20%. The UV absorbing agent would be present in an amount effective to prevent degradation of hinokitiol more than 50% after exposure to sunlight for more than 5 hours. The UV protection agents include but are not limited to L-ascorbic acid, humic acid, and sodium salt of oxylignin or calcium lignosulfonate.

The compositions of the present invention may further comprise a basic pH stabilizer. As defined herein, a basic pH stabilizer is a substance that maintains the pH of the composition of the present invention between 7-10. pH stabilizing agents include but are not limited to metal salts and alkali hydroxides listed on EPA Lists 4A and 4B. These include but are not limited to sodium/potassium bicarbonate, sodium/potassium carbonate, sodium/potassium hydroxide, sodium/potassium acetate, sodium/potassium citrate.

The compositions of the invention do comprise a carrier and/or diluent. The term, ‘carrier’ as used herein means an inert, organic or inorganic material, with which the active ingredient is mixed or formulated to facilitate its application to the soil, seed, plant or other object to be treated, or its storage, transport and/or handling. Examples of carrier vehicles to be used when applying to growth substrates include, but are not limited to, active charcoal, corn gluten meal, soybean meal, vermiculite, bentonite, kaolinite, wheat germ, almond hulls, cottonseed meal, Fuller's earth, orange pulp, rice hulls, sawdust, Gum arabic, etc. If desired, plant essential oils such as cinnamon, clove, thyme (eugenol as active ingredient), wintergreen, soy methyl ester, citronella and pine oil, citrus oil (1-limonene as active ingredient) and the like, can be included in the granules. As noted above, the active ingredient alone or in the presence of the carrier vehicles, may be dissolved in for example, water, or organic solvent such as ethanol, formic acid or ethanol. The composition may further comprise an additional fungicidal agent such as Bacillus subtilus, myclobutanil, and fenhexamide, azoxystrobin, azoxystrobin combination, boscalid, bacillus subtilis, copper sulfate, chlorothalonil, copper hydroxide, cymoxanil, dimethomorph, dechloropropene, fosetyl-aluminum, fludioxonil, fenamidone, iprodione, mefenoxam, mancozeb, metalaxyl, metam sodium, potassium bicarbonate, pyraclostrobin, propiconazole, propicocarb, thiram, thiabendazole, thiophanate-methyl, trifloxystrobin, vinclozolin, sulfur, ziram.

The carrier used may depend on whether it is being used in a pre- or post-emergence herbicide. Liquid carriers can be used for both pre and post-emergence applications. Examples of carrier vehicles for the pre-emergent herbicide include, but are not limited to, active charcoal, corn gluten meal, soybean meal, vermiculite, bentonite, kaolinite, wheat germ, almond hulls, cottonseed meal, Fuller's earth, orange pulp, rice hulls, sawdust, Gum arabic, etc. If desired, plant essential oils such as cinnamon, clove, thyme (eugenol as active ingredient), wintergreen, citronella and pine oil, and the like, can be included in the granules to improve the pre-emergent and post-emergent effect of hinokitiol. Examples of diluents or carriers for the post-emergence herbicides include, but are not limited to, water, milk, ethanol, mineral oil, glycerol, and other organic acids, particularly aliphatic carboxylic acids (e.g., C1-C10 carboxylic acid) such as formic acid, acetic acid or propionic acid.

The composition may additionally comprise a surfactant to be used for the purpose of emulsification, dispersion, wetting, spreading, integration, disintegration control, stabilization of active ingredients, and improvement of fluidity or rust inhibition. In a particular embodiment, the surfactant is a non-phytotoxic non-ionic surfactant which preferably belongs to EPA List 4B. In a particular embodiment, the nonionic surfactant is polyoxyethylene (20) monolaurate. The concentration of surfactants may range between 0.1-35% of the total formulation, preferred range is 5-25%. The choice of dispersing and emulsifying agents, such as non-ionic, anionic, amphoteric and cationic dispersing and emulsifying agents, and the amount employed is determined by the nature of the composition and the ability of the agent to facilitate the dispersion of the herbicidal compositions of the present invention. In a particular embodiment, the composition is free of an amine containing surfactant.

For pre-emergence dry formulations, the granule size of the carrier is typically 1-2 mm (diameter) but the granules can be either smaller or larger depending on the required ground coverage. Granules may comprise of porous or non-porous particles.

For post-emergence formulations, the formulation components used may contain smectite clays, attapulgite clays and similar swelling clays, thickeners such as xanthan gums, gum Arabic and other polysaccharide thickeners as well as dispersion stabilizers such as nonionic surfactants (for example polyoxyethylene (20) monolaurate). The concentration of surfactants may range between 0-25% of the total formulation. The concentration of the clays may vary between 0-2.5% w/w of the total formulation, the polysaccharide thickeners may range between 0-0.5% w/w of the total formulation and the surfactants may range between 0-5% w/w of the total formulation. In a particular embodiment, the formulation may comprise about 17.0-19.0% dispersing agent, 26.0-30.0% water, 0.005-1.5% emulsifier and 53% hinokitiol (60% in propionic acid).

The composition and method of the present invention will be further illustrated in the following, non-limiting examples. The examples are illustrative of various embodiments only and do not limit the claimed invention regarding the materials, conditions, weight ratios, process parameters and the like recited herein.

### EXAMPLES

The composition and method of the present invention will be further illustrated in the following, non-limiting Examples. The examples are illustrative of various embodiments only and do not limit the claimed invention regarding the materials, conditions, weight ratios, process parameters and the like recited herein.

### Example 1

The spiral gradient dilution method (Forster, H., Kanetis, L., Adaskaveg, J. E. 2004. Spiral gradient dilution, a rapid method for determining growth responses and 50% effective concentration values in fungus—fungicide interactions. Phytopathology 94: 163-170) was used for determination of 50% effective concentrations (EC₅₀ values) of hinokitiol for the inhibition of mycelial growth and spore/conidial germination of various plant pathogenic fungi. In this method, a potato dextrose agar (PDA) medium is plated with the test solution by means of a spiral plater, which applies the compound in a 2.5-log dilution in a continuous radial concentration gradient. Fungal inoculum in two replicates is then placed along radial lines across the gradient. After one day of incubation at 20° C., plates were observed for inhibition of spore germination and the following day, measurements were taken for distinct growth shapes observed in different fungus-hinokitiol interactions. EC₅₀ values were calculated based on these measurements. The fungi evaluated were members of the following families Anamorphic Phyllachoraceae (Colletotrichum acutatum), Anamorphic Sclerotiniaceae (Botrytis cinerea), Mitosporic fungi (Verticillium dahlias; Microphomina sp.), and Oomycota (Phytophtora capsici). Hinokitiol (5 mg/mL) was dissolved in acetone, and acetone alone was also used as a control to check for non-specific inhibition.

When the hinokitiol plates inoculated with B. cinerea were examined under the 10× objective of a light microscope, it was noted that hinokitiol at concentrations 5-0.05 mg/mL resulted in a 100% inhibition in spore germination. The EC₅₀ values calculated based on the growth measurements are listed in Table I:

TABLE I

| Inoculum | EC₅₀ (mg/mL) |
|---|---|
| Colletotrichum acutatum | 0.012 |
| Botrytis cinerea | 0.031 |
| Verticillium dahliae | 0.011 |
| Microphomina sp. | 0.011 |
| Phytophtora capsici | 0.012 |

These results indicate that hinokitiol is a potential biofungicide for controlling plant pathogenic fungi, and it is effective at very low concentrations.

### Example 2

Another set of hinokitiol spiral PDA plates prepared as above were inoculated with plant pathogenic fungi from the genera Mucor, Rhizoctonia, Geotrichum, Phoma, and Penicillium. After 2 days of incubation, growth was only observed in the plates treated with acetone alone, which indicates a complete inhibition of the test fungi by hinokitiol.

### Example 3

To further test the inhibitory effect of hinokitiol on grey mold (Botrytis cinerea), an in vivo test on green bell pepper (Capsicum annuum) was conducted in a greenhouse. Pepper plants at a 6-true-leaf growth stage were sprayed with hinokitiol in 50% ethanol. The stock solution (5 mg/mL) was prepared in 97% ethanol, and diluted to following concentrations: 0, 0.01, 0.1 mg/mL. To compare the efficacy of hinokitiol in an alcohol-free solution, a treatment with 0.1 mg/mL of hinokitiol in water was included in this study. Pepper plants with no treatment were used as controls. All treatments were carried out in 3 replicates. After treatment with hinokitiol, the leaf surfaces were allowed to dry under the growth lights, after which the plants were inoculated with a B. cinerea spore solution containing 2×10⁶ spores per mL. Inoculated plants were incubated in the dark in a covered flat at 22° C. Plants were observed at 2, 3, and 7 days after treatment for disease symptoms. Symptoms were rated on a scale of 0-5; 0 indicating no lesions on leaves, and 5 indicating severe rotting/dead plant.

Results of this in vivo test are listed in Tables II and III below:

TABLE II

| | | | | rating |
|---|---|---|---|---|
| Treatment | Replicate | rating at day 2 | rating at day 3 | at day 7 |
| Untreated control | 1 | 2 | 3 | 5 |
| | 2 | 3 | 4 | 5 |
| | 3 | 3 | 4 | 5 |
| Ethanol 50% | 1 | 3 | 5 | 5 |
| | 2 | 2 | 3 | 3 |
| | 3 | 2 | 4 | 5 |
| Hinokitiol at | 1 | 2 | 4 | 5 |
| 0.01 mg/mL in | 2 | 2 | 3 | 3 |
| 50% ethanol | 3 | 1 | 1 | 1 |
| Hinokitiol at | 1 | 2 | 3 | 5 |
| 0.1 mg/mL | 2 | 1 | 2 | 5 |
| in 50% ethanol | 3 | 2 | 3 | 2 |
| Hinokitiol at | 1 | 0 | 0 | 1 |
| 0.1 mg/mL | 2 | 1 | 1 | 2 |
| in water | 3 | 0 | 1 | 4 |

TABLE III

| Treatment | 2 days | 3 days | 7 days |
|---|---|---|---|
| Untreated | 2.7 a | 3.7 ab | 5.0 a |
| Ethanol | 2.3 a | 4.0 a | 4.3 ab |
| Hinokitiol 0.01 mg/mL EtOH | 1.7 a | 2.7 ab | 3.0 ab |
| Hinokitiol 0.1 mg/mL EtOH | 1.7 a | 2.7 ab | 4.0 ab |
| Hinokitiol 0.1 mg/mL H₂O | 0.3 b | 0.7 b | 2.3 b |
| LSD (Least square differences) (0.05) | 1.07 | 1.7 | 2.66 |

Average (means) followed by the same letter within columns are not significantly different at P = 0.05 according to least significant difference (LSD) test. Averages followed by a different letter are statistically different.

The incidence of B. cinerea was significantly lower in plants treated with hinokitiol (0.1 mg/mL water) than in the untreated control pepper plants. Hinokitiol dissolved in an organic solvent (ethanol) at rate of 0.01 and 0.1 mg/mL postponed the disease outbreak but the final disease incidence was not significantly different from the untreated control.

### Example 4

Numerous natural compounds were screened for their ability to inhibit the germination of dicot (broadleaved weed) seeds. A single seed of Lactuca sativa (lettuce) was placed in each well of a 96-well plate followed by 50 μL of a solution of each compound in a stepwise (5×) dilution series from 25% to 0%. Germination was monitored daily. Based on this screening study, the threshold value for hinokitiol to inhibit germination of seeds was determined at 0.32 mg/L.

### Example 5

A high-throughput 96-well assay was used to test the efficacy of hinokitiol as a post-emergence, non-selective herbicide. Seedlings of Lactuca sativa (lettuce) were grown in 96-well plates under continuous light. Hinokitiol was added on the one-week old seedlings at a 5×-dilution series from 1 to 0 mg/mL, and the minimum concentration needed for killing the seedling was recorded the next day. According to the results, hinokitiol at a concentration of 40 mg/L was able to kill the lettuce seedlings where as hinokitiol at a concentration of 8 mg/L was not harmful to the plant.

### Example 6

A pot study was conducted to test the phytotoxicity of hinokitiol on both broadleaved and grass weed. Ten seeds of either chickweed (Stellaria media) or annual bluegrass (Poa annua) were planted in a plastic pot filled with potting mix. The 2-inch tall plants grown under growth lights (12-h light/12-h dark) at room temperature were sprayed with hinokitiol solutions containing 0.5, 1.0, 2.0, 3.0, 4.0 and 5.0 mg/mL hinokitiol in 50% ethanol. A solution of 50% ethanol without hinokitiol was used as a control treatment. The plants were kept at room temperature under growth lights and observed at two time points—4 and 14 days after treatment—for visual symptoms of phytotoxicity and % weed control.

Symptoms of phytotoxicity were visible in the plants treated with solutions of high hinokitiol content one day after the treatment. The % weed control obtained with different concentrations of hinokitiol is listed in Table IV, infra.

TABLE IV

| Hinokitiol | Stellaria media | | | |
|---|---|---|---|---|
| (mg/mL in | (% control) | | Poa annua (% control) | |
| 50% ethanol) | 4 DAYS | 14 DAYS | 4 DAYS | 14 DAYS |
| 0 | 0 | 0 | 0 | 0 |
| 0.5 | 1 | 0 | 0 | 0 |
| 1.0 | 2 | 10 | 1 | 0 |
| 2.0 | 10 | 55 | 2 | 5 |
| 3.0 | 95 | 100 | 10 | 15 |
| 4.0 | 98 | 100 | 30 | 25 |
| 5.0 | 100 | 100 | 50 | 50 |

### Example 7

In the instant example, the efficacy of hinokitiol in controlling Botrytis gray mold affecting pepper is determined.

Pepper plants (California Wonder Bell Pepper) were treated with a 3.2% Hinokitiol formulation containing propylene glycol, nonphytotoxic ethoxylate fatty esters; butanol, EDTA, UV protection agent, 1% of a 15% solution of sodium hydroxide and water 3 hours before and 24 hours after inoculation (pre and post infection, respectively). Hinokitiol concentration in pre-infection application was 1.0% v/v and for post-infection application was 2.0% v/v. Three plants were included per treatment in pre-infection applications, and 18 plants in post-infection treatments. The formulation employed contained 3.2% of active ingredient. Inoculation was performed with a conidial suspension of 1.8×10⁶ conidia per ml of 75% PDB (Potato Dextrose Broth). Scale of evaluation was 0=no infection, 1=Small scattered lesions, 2=Scattered big lesions or small ones covering more than 50% of leaf surface, 3=Half plant dead, 4=75% of the plant dead, 5=Entire plant dead. Evaluation was performed 3 days after inoculation.

The results are shown in Tables V and VI below. Table V shows the intensity of disease based on the scale of severity described in materials and methods. Hinokitiol (0.32 mg/ml) applied 3 hr before inoculation and Table VI shows shows the number of pepper plants infected with Botrytis gray mold after treatment with hinokitiol (0.64 mg/ml) 24 hr after inoculation.

TABLE V

| Treatment | Plant 1 | Plant 2 | Plant 3 |
|---|---|---|---|
| UTC not inoculated | 1 | 1 | 1 |
| UTC inoculated | 5 | 5 | 5 |
| Hinokitiol 1.0% | 1 | 1 | 1 |

1 = Small scattered lesions;
5 = Entire plant dead;
UTC = untreated control

TABLE VI

| | | Total | Scale of infection | | | | | |
|---|---|---|---|---|---|---|---|---|
| Treatment | Rep | plants | 0 | 1 | 2 | 3 | 4 | 5 |
| UTC | 1 | 18 | 0 | 0 | 0 | 0 | 0 | 18 |
| | 2 | 18 | 1 | 4 | 2 | 2 | 0 | 9 |
| Hinokitiol | 1 | 18 | 0 | 1 | 12 | 4 | 1 | 0 |
| | 2 | 18 | 1 | 6 | 7 | 1 | 2 | 1 |

1 = Small scattered lesions;
5 = Entire plant dead;
UTC = untreated control

As shown in Table V, pepper plants treated with hinokitiol formulations at 1% (0.32 mg/ml) dilution did not show any visible syptoms of disease or phytotocicity. It appears that this formulation at an application rate of 1% (100 dilution) can effectively fight against the grey mold inoculation. For curative purposes, as shown in Table VI, a concentration of 2% (0.64 mg/ml) might provide better efficacy than the 1% solution.

### Example 8

In the instant example, the herbicidal effect of hinokitiol in a field study is determined.

A hinokitiol formulation containing 32.4% of hinokitiol by weight was tested in a field study. The formulation also contained dispersing agent, water and emulsifier. The two concentrations tested contained 5% and 0.5% hinokitiol (w/w), and the spraying volume was 100 gallons per acre. Three replicate 1-square-foot plots were sprayed with the appropriate solutions, and the % weed control in each plot was recorded at 3, 7, 14 and 20 days after treatment. The main weeds in this study were spurge, white clover, annual bluegrass and crab grass. The results are shown in Table VII infra.

TABLE VII

| | % weed control | | | |
|---|---|---|---|---|
| Hinokitiol % | 3 DAYS | 7 DAYS | 14 DAYS | 20 DAYS |
| 5.0 | 86.7 | 90.3 | 88.3 | 83.7 |
| 0.5 | 3.3 | 3.7 | 1.7 | 3.0 |

As shown in Table VII, one week after spraying, the hinokitiol formulation containing 5% (w/w) hinokitiol controlled more than 90% of all weeds. However, the herbicidal effect of the same solution at 10× lower concentration had only a negligible effect on weeds.

### Example 9

In the instant example, the preparation of hinokitiol formulation and testing of various components of a hinokitiol formulation is described.

### Non-Ionic Surfactants

Ethoxylated fatty esters (e.g., polyethylene 20 sorbitan monooleate, polyethylene 20 sorbitan monolaurate), ethoxylated alcohols (e.g., Genapol UD-80, UD 110, O-100 and O-200, Clariant Corporation) were chosen for phytotoxic screening in this study. They belong to the EPA List 4B. All tested formulations contained co-solvents (15%), water (55%) and surfactant (30%). These formulations were diluted 100 times with tap water and then sprayed on 2-week-old cucumber plants. The results indicated that all ethoxylated alcohol surfactants displayed phytotoxicity. Between the two ethoxylated fatty esters, polyethylene 20 sorbitan monooleate did not show any obvious phytotoxicity, but polyethylene 20 sorbitan monolaurate did.

### Chelators

In this study, citric acid (List 4A), citric acid disodium salt (List 4A) and EDTA disodium salt (List 4B) were chosen for testing.

Formulation compositions: All formulations contained 3.2% hinokitiol, 2% propylene glycol, 20% polyethylene 20 sorbitan monooleate, 0.5% propyl 4-hydroxybenzoate, 2% 1-butanol, a certain amount of chelator and water for a total of 100%. Percent concentrations of citric acid, citric acid disodium salt and EDTA disodium salt were 3.2%, 3.2% and 1.6%, respectively. The total weight of each formulation was 50 grams.

Preparation procedures: Step 1: dissolved hinokitiol and propyl 4-hydroxybenzoate in the mixture of 1-butanol, propylene glycol and polyethylene 20 sorbitan monooleate under warm water bath; Step 2: added this mixture to vigorous stirring water to form oil-in water microemulsion. The final product is transparent solution; Step 3: divided each formulation evenly into three groups for storage tests.

Storage procedures: Standard accelerated storage test (CIPAC) were applied with some modifications. Briefly, formulations were put into 20 mL glass scintillation vials. The caps were sealed with black sticky bands. The vials were separately stored in a 4° C. refrigerator and a 54° C. incubator. Vials with formulations were taken out and visual observation was done as soon as possible.

HPLC analysis: HPLC systems include waters 2695 separation modules with auto sampler, waters 2996 photodiode array detector, Masslynx software and Sunfire™ C18 column (5 μm, 4.6×150 mm). Mobile phase included solvent A (0.5% citric acid in water) and solvent B (0.1% trifluoroacetic acid in acetonitrile). Gradient mobile phase was adopted. The solvent B remained 20% in the first 4 min, increased from 20% to 80% in the next 4 min, then remained 80% for 4 min and then decreased to 20% with 8 min. Hinokitiol complex with citric acid was monitored at 320 nm. The column temperature was kept at 30° C. during the experiment.

Sun exposure experiment: 0.5 mL sample was added into 3-ml vials and were capped. The vials were put on a rack. The rack was put under sun light to make sure each vial obtains the same sun exposure (vials were leaning against the rack with an angle of 30-50 degree); After sunlight exposure, rack and vials were immediately stored at 4° C. or subsamples were taken for HPLC analysis. Visual observation was recorded before subsamples were taken. Vials were vortexed very well before subsamples were taken. Triplicates were carried out for each sample. All sun light exposure in the later studies was the same unless stated. Temperature in all this studies varied between 75 to 95° F.

The results are shown in Table VIII and indicate that all examined chelators stabilized hinokitiol within 4 to 54° C. for two weeks, which met our goal based on the accelerated storage tests (CIPAC method). There were no significant differences in the hinokitiol stability among these chelators.

TABLE VIII

| | | Temperature | | |
|---|---|---|---|---|
| | | (14 days) | Sun exposure (7 h) | |
| | | degradation of | Degradation of | |
| | Observation | hinokitiol | hinokitiol | |
| Chelator | (14 days) | (%) | (%) | Visual observation |
| W/O | Crystallized (4° C.) | ND | ND | ND |
| | Crystallized | ND | ND | ND |
| | (54° C.) | | | |
| Citric acid | Uniform (4° C.) | 1 ± 3 (4° C.) | 78 ± 3 | From slight |
| | Uniform | 2 ± 2 (54° C.) | | yellowish became |
| | (54° C.) | | | dark brown |
| Citric acid | Uniform (4° C.) | 0 ± 1 (4° C.) | 81 ± 2 | From slight |
| disodium salt | Uniform | 1 ± 2 (54° C.) | | yellowish became |
| | (54° C.) | | | dark brown |
| EDTA-disodium | Uniform (4° C.) | 0 ± 1 (4° C.) | 78 ± 3 | From slight |
| salt | Uniform | 0 ± 1 (54° C.) | | yellowish became |
| | (54° C.) | | | dark brown |

ND: Not determined.

### UV Protection Agents

There are many UV protection agents reported in literature and patents. For example, organic UV absorbers and polymeric UV absorbers were disclosed in the patent WO2007077259. Organic UV absorbers consist of 4-aminobenzoic acid and derivatives, salicylic acid derivatives, benzophenone derivatives, dibenzoylmethane derivatives, diphenyl acrylates, 3-imidazol-4-yl-acrylic acid and esters thereof, benzofuran derivatives, benzylidenemalonate derivatives. Polymeric UV absorbers contain one or more organosilicon radicals, cinnamic acid derivatives, camphor derivatives, trianilino-s-triazine derivatives, 2-hydroxyphenylbenzotriazole derivatives, phenylbenzimidazolesulfonic acid derivatives and salts thereof, anthranilic acid menthyl ester, benzotriazole derivatives, indole derivatives. However, none of these are listed in the US EPA Lists 4A and 4B. Therefore, all compounds that are listed on the EPA Lists 4A and 4B were chosen for this study. They consisted of zinc oxide, titanium dioxide, L-ascorbic acid, humic acid, bentonite, sodium salt of oxylignin and calcium lignosulfonate.

Formulation compositions: All formulations contained 3.2% hinokitiol, 2% propylene glycol, 20% polyethylene 20 sorbitan monooleate, 2% 1-butanol, 1.6% EDTA disodium salt, a certain amount of UV protection agent and water for a total of 100%. The total weight of each formulation was 20 grams.

All samples were prepared in a similar method as described above. After each formulation was stored at least overnight at 4 or 54° C., subsamples were taken for sun light exposure tests (procedure as described above). A second sample (0.5 mL) of each formulation was taken for pH analysis. The remaining formulation was divided evenly into two parts, which were separately stored 4 and 54° C. for further storage observation. pH analysis was monitored by pH/mV/thermometer (IQ Scientific Instruments) after 100-fold dilution with a standard hard water (342 ppm). Visual observations for layer, precipitation and/or crystallization were recorded every day for storage tests.

The results shown in Table IX below indicate that metal oxides (e.g., ZnO and TiO₂) and metal complex (e.g., bentonite) did not dissolve into the formulation even at 0.1%. Organic acids (L-ascorbic acid humic acid) and salts (e.g., sodium salt of oxylignin and calcium lignosulfonate) could be added into the formulations, but the degradation of hinokitiol in the formulation was much slower in the humic acid and sodium salt of oxylignin.

TABLE IX

| | | | Length of | Degradation | |
|---|---|---|---|---|---|
| UV protection | | | sunlight | of hinokitiol | |
| agent | % content | pH | exposure (h) | (%) | Note |
| ZnO | 0.1 | ND | ND | ND | Precipitate after 2-hr |
| | 0.2 | ND | ND | ND | storage |
| TiO₂ | 0.1 | ND | ND | ND | Precipitate after 2-hr |
| | 0.2 | ND | ND | ND | storage |
| Bentonite | 0.25 | ND | 6 | 61 ± 3 | Precipitate after 2-day |
| | 0.5 | ND | 6 | 59 ± 3 | storage |
| L-ascorbic acid | 0.5 | ND | 6 | 52 ± 4 | |
| | 1.0 | ND | 6 | 56 ± 2 | |
| Humic acid | 15 | 7.40 | 16 | 40 ± 1 | A slight insoluble |
| | | | | | particulate in the |
| | | | | | bottom after 2-day |
| | | | | | storage at 54° C. |
| Oxylignin | 10 | 6.71 | 16 | 47 ± 2 | High viscosity for 20% |
| (sodium salt) | 20 | 7.09 | 16 | 13 ± 7 | Oxylignin (sodium |
| | | | | | salt) |
| Calcium | 20 | 3.96 | 16 | 79 ± 4 | A large amount of |
| lignosulfate | | | | | particulates |
| | | | | | precipitated out after |
| | | | | | 5-day storage |

ND: Not determined

### pH Adjustment

Formulation compositions: All formulations contained 3.2% hinokitiol, 2% propylene glycol, 20% polyethylene 20 sorbitan monooleate, 2% 1-butanol, 1.6% EDTA disodium salt, 20% Oxylignin (sodium salt) (or 15% humic acid), a certain amount of sodium hydroxide and water for a total of 100%. The total weight of each formulation was 20 grams.

All samples were prepared in a similar method as described above. After each formulation was stored at least overnight at 4 or 54° C., subsamples were taken for sun light exposure tests (procedure as described above). A second sample (0.5 mL) of each formulation was taken for pH analysis. The remaining formulation was divided evenly into two parts, which were separately stored 4 and 54° C. for further storage observation. pH analysis was monitored by pH/mV/thermometer (IQ Scientific Instruments) after 100-fold dilution with standard hard water. Visual observations for layer, precipitation and/or crystallization were recorded every day for storage tests.

Results (Table X) suggested that pH played a great role on the stability of hinokitiol in the microemulsion. Precipitation would happen to Oxylignin (sodium salt) when pH reached to 8.0 and then increased with pH. In addition, severe phytotoxicity on 2-week-old cucumber plants was shown when pH reached 8.7. In contrast, there was no obvious phytotoxicity on 2-week-old cucumber plants for humic acid at pH 10.

TABLE X

| | % content of | | | | |
|---|---|---|---|---|---|
| UV | UV | Length of | | Degradation | |
| protection | protection | sun | | of hinokitiol | |
| agent | agent | exposure (h) | pH | (%) | Note |
| Oxylignin | 20 | 16 | 7.09 | 40 ± 1 | Precipitate increased |
| (sodium salt) | 20 | 16 | 8.40 | 24 ± 4 | with pH; and displayed |
| | | | | | phytotoxicity at pH |
| | | | | | over 8.7 |
| Humic acid | 15 | 16 | 7.40 | 40 ± 3 | A slight insoluble |
| | 15 | 16 | 8.29 | 30 ± 2 | particulate in the |
| | 15 | 16 | 9.61 | 21 ± 4 | bottom after 14-day |
| | | | | | storage at 54° C. |

### Analysis of Hinokitiol Residue on Green Beans After Sun Exposure

The goal of this study was to know stability of hinokitiol on plant leaves, instead of vials, after sun exposure. The following procedure was used
1. A 3.2% Hinokitiol formulation containing propylene glycol, nonphytotoxic ethoxylate fatty esters; butanol, EDTA, UV protection agent, 1% sodium hydroxide and water for a total of 100% was used. The total weight of each formulation was 20 grams.
2. Spraying solution: 10 mL of 100-fold dilution of the above formulation.
3. Green bean plants: 6 plants with 10-15 leaves.
4. Two plants were evenly sprayed 10 mL solution.
5. Three plants was stored in dark incubator (25° C.), another three plants was put on the parking lot with full sun exposure (28˜35° C.).
6. After 6 h, leaves were cut and punched into the same size discs (Ø21 mm). Discs from sun-exposed green bean and darkness green bean were 118 and 129, respectively.
7. Discs were ground in a mortar and transferred into a separation funnel. The mortar and pestle were sequentially washed with 2 N HCl and ethyl acetate. The hinokitiol was extracted three times (3×50 mL) by ethyl acetate under 2N HCl (100 mL). The organic phase was dried by anhydrous sodium sulfate. After filtration and evaporation, the organic phase gave out a residue. The residue was dissolved by ethanol (1.5 mL). This solution was directly used for HPLC analysis. Injection volume was 10 μL.

### Data for Calculation

Darkness: leaf disc 129
Sun exposure: leaf disc 118
Darkness: Peak area (AU): 50463
Sun exposure: Peak Area (AU); 13197
Standard line: y=66.367X-52.019 (R²=0.9999, X=μg/ml of hinokitiol standard solution, 2 μL injection volume; y=peak area)

### Calculation of Hinokitiol Concentration in the HPLC Injection Solution

[hinokitiol] from darkness=[(50463+52.019)/66.367]/(10/2)=152 μg/mL
[hinokitiol] from sun exposure=[(13197+52.019)/66.367]/(10/2)=40 μg/mL

### Calculation of Hinokitiol Concentration on Each Disc of Green Beans

[hinokitiol] from darkness=152 μg/mL*1.5 mL/129 Disc=1.77 μg/Disc
[hinokitiol] from sun exposure=40 μg/mL*1.5 mL/118 Disc =0.51 μg/Disc

### Relative Stability:

[hinokitiol] from sun exposure/[hinokitiol] from darkness=0.51/1.77=0.29

Although background noise was high compared with standards, the hinokitiol peak for both darkness and sun exposure was still three times higher than the background noise. Of course, this high background would decrease the detection limit of hinokitiol and also affected the actual concentration of hinokitiol. If further study is needed, cleaning up is supposed to be performed before the HPLC analysis.

However, after 6 h sun exposure at 28-35° C., hinokitiol concentration per unit area (Disc) on green bean plants was still 29% of that stored in the darkness at 25° C. This means that the residue on this plant would last at least one day.

### Example 10

This example describes tests on the effect of hinokitiol in acetone on the growth of Monilinia fructicola (MON), Botrytis cinerea (BOT), Fusarium sp (FUSM), Xanthomonas campestris (X-CAM), Xanthomonas vesicatoria (X-VES), Pseudomonas viridilivida (PSVI), and Erwinia carotovora (ERWC).

### Method

The spiral plating method was used to deposit the fungicides on PDA and TSA plates with exponential logarithmic decrease of product concentration from the centre to the edge of the plate. The plates were left standing for four hours and inoculated with the fungus and bacteria. There were four repetitions of the same microorganism done per plate and there was one replica of each plate.

The plates were cultured for 3 days before measurements of growth were taken. The ‘distance of inhibition’ indicates the length of the zone with no growth, measured from center edge of the agar, the ‘colony width ratio’ indicates the ratio of the width of growth measured at the zero concentration and width at the highest concentration with growth.

### Results

The results are shown in Table XI.

TABLE XI

| | | Distance of | Colony width | Distance of | Colony width |
|---|---|---|---|---|---|
| | | inhibition (4 | ratio | inhibition (4 | ratio |
| Media | Pathogen | Reps, plate 1) | plate 1 | Reps, plate 2) | plate 2 |
| TSA | X-CAM | 1.-11 mm | 1:1 | 1.-28 mm | * |
| | | 2.-28 mm | * | 2.-28 mm | * |
| | | 3.-18 mm | 1:1 | 3.-28 mm | * |
| | | 4.-17 mm | 1:1 | 4.-28 mm | * |
| TSA | X-VES | 1.-28 mm | * | 1.-28 mm | * |
| | | 2.-28 mm | * | 2.-28 mm | * |
| | | 3.-28 mm | * | 3.-28 mm | * |
| | | 4.-28 mm | * | 4.-28 mm | * |
| TSA | PSVI | 1.-0 mm | 1:1 | 1.-0 mm | 1:1 |
| | | 2.-0 mm | 1:1 | 2.-17 mm | 4:1 |
| | | 3.-0 mm | 1:1 | 3.-0 mm | 1:1 |
| | | 4.-0 mm | 1:1 | 4.-0 mm | 1:1 |
| TSA | ERWC | 1.-16 mm | 1:1 | 1.-28 mm | * |
| | | 2.-16 mm | 5:1 | 2.-28 mm | * |
| | | 3.-8.5 mm | 5:1 | 3.-28 mm | * |
| | | 4.-11 mm | 5:2 | 4.-4 mm | 5:2.5 |
| PDA | MON | 1.-0 mm | 1:1 | 1.-13 mm | 17:3 |
| | | 2.-28 mm | * | 2.-11 mm | 16:3 |
| | | 3.-0 mm | 1:1 | 3.-16.5 mm | 6:2 |
| | | 4.-28 mm | * | 4.-12 mm | 10:5 |
| PDA | BOT | 1.-0 mm | 1:1 | 1.-0 mm | 1:1 |
| | | 2.-0 mm | 1:1 | 2.-0 mm | 1:1 |
| | | 3.-0 mm | 1:1 | 3.-0 mm | 1:1 |
| | | 4.-0 mm | 1:1 | 4.-0 mm | 1:1 |
| PDA | FUSM | 1.-2 mm | 1:1 | 1.-8 mm | 11:3 |
| | | 2.-18 mm | 1:1 | 2.-6 mm | 1:1 |
| | | 3.-14 mm | 1:1 | 3.-6 mm | 1:1 |
| | | 4.-9.5 mm | 1:1 | 4.-8 mm | 1:1 |

0 mm of growth inhibition = everything grew, no inhibition;
28 mm of growth inhibition = maximum inhibition, no growth;
* Means there was no colony to measure. The % inhibition was calculated by diving the average distance of inhibition by the distance indicating 100% inhibition (28 mm) × 100.

Hinokitiol inhibited the growth of X. campestris by 83%, E. carotovora by 62%, M. fructicola by 48%, and Fusarium sp by 32%. Hinokitiol did not inhibit the growth of P. viridilivida and B. cinerea. With E. carotovora and M. fructicola the fungal and bacterial horizontal growth decreased as the concentration of hinokitiol increased.

Although this invention has been described with reference to specific embodiments, the details thereof are not to be construed as limiting, as it is obvious that one can use various equivalents, changes and modifications and still be within the scope of the present invention.

Various references are cited throughout this specification, each of which is incorporated herein by reference in its entirety.

What is claimed is:

1. A composition comprising hinokitiol, salt thereof, or combination of the foregoing, an acid chelator and a carrier and/or diluent.

2. The composition wherein said composition comprises hinokitiol.

3. The composition according to claim 1, wherein said composition comprises a hinokitiol salt.

4. The composition according to claim 1, wherein said hinokitiol salt thereof, or combination of the foregoing is in an organic solvent.

5. The composition according to claim 1, wherein said hinokitiol salt thereof, or combination of the foregoing is in an organic solvent selected from the group consisting of formic acid, propionic acid, ethanol, butanol and acetone.

6. The composition according to claim 1, wherein said composition is in the form of an emulsifiable concentrate, microemulsion, soluble liquid, emulsion oil-in-water, suspension concentration, wettable granule, or wettable powder.

7. The composition according to claim 1, wherein said composition further comprises a non-hinokitiol antifungal agent.

8. The composition according to claim 1, wherein said composition further comprises a non-hinokitiol bioherbicidal or non-hinokitiol chemical herbicidal agent.

9. The composition according to claim 1, wherein said chelator is selected from the group consisting of EDTA, citric acid and a citric acid salt.

10. The composition according to claim 1, wherein said composition further comprises a non-phytotoxic nonionic surfactant.

11. The composition according to claim 10, wherein said composition further comprises a basic pH stabilizing agent.

12. The composition according to claim 11, wherein said composition comprising said pH stabilizing agent has a pH of 7-10.

13. The composition according to claim 11, wherein said composition further comprises a UV protection agent.

14. The composition according to claim 13, wherein said UV protection agent is selected from the group consisting of L-ascorbic acid, humic acid, bentonite, sodium salt of oxylignin and calcium lignosulfonate.

15. A composition comprising hinokitiol, an acid chelator, a non-phytotoxic nonionic surfactant, butanol, propylene glycol, an acid chelator, and a UV protection agent, pH stabilizing agent and a diluent and/or carrier.

16. The composition according to claim 15, wherein the pH stabilizing agent is a metal hydroxide.

17. A method for modulating non-Erwinia microbial infection in a plant comprising applying to said plant and/or seeds thereof and/or substrate used for growing said plant an amount of hinokitiol and/or salt thereof to modulate non-Erwinia, non-Pseudomonas microbial infection in said plant.

18. A method for modulating fungal infection in a plant comprising applying to said plant and/or seeds thereof and/or substrate used for growing said plant the composition of claim 1 to modulate non-Erwinia, non-Pseudomonas microbial infection in said plant.

19. The method according to claim 18, wherein said growth substrate is soil.

20. The method according to claim 18, wherein said composition is applied prior to harvest of said plant.

21. The method according to claim 18, wherein said non-Erwinia microbial infection is fungal infection.

22. The method according to claim 21, wherein said fungal infection is a Fusarium sp., Botrytis sp., Monilinia sp., Colletotrichum sp, Verticillium sp.; Microphomina sp., and Phytophtora sp, Mucor, Rhizoctonia, Geotrichum, Phoma, or Penicillium infection.

23. The method according to claim 18, wherein said non-Erwinia microbial infection is bacterial infection.

24. The method according to claim 18, wherein said non-Erwinia microbial infection is Xanthomonas infection.

26. A method for modulating growth of monocotyledonous or dicotyledonous weeds comprising applying to said weeds an amount of hinokitiol effective to modulate growth of said weeds.

27. A method for modulating emergence of monocotyledonous or dicotyledonous weeds in soil comprising applying to said weeds or soil an amount of the composition of claim 1 effective to modulate emergence of said weeds.

28. The method according to claim 27, wherein said weeds are broadleaved and/or grass weeds.
